(19) United States
(12) Patent Application Publication
(10) Pub. No.: US 2009/0192594 A1
(43) Pub. Date: Jul. 30, 2009

(54) IMPLANT HAVING A BASE BODY OF A BIOCORRODIBLE ALLOY AND A CORROSION-INHIBITING COATING

(75) Inventor: Alexander Borck, Aurachtal (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/362,129

(22) Filed: Jan. 29, 2009

(30) Foreign Application Priority Data
Jan. 29, 2008 (DE) 10 2008 006 455.6

(51) Int. Cl. A61F 2/82

(52) U.S. Cl. 623/1.46

(57) ABSTRACT

An implant having a base body consisting entirely or in part of a biocorrodible metallic material wherein at least the parts of the base body made of the biocorrodible metallic material are covered with a corrosion-inhibiting coating. The coating comprises a primer layer of a first biodegradable polymer material comprising a poly(D,L-lactide) (PDLLA) with a degree of polymerization in the range of 5 to 20 and a protective layer of a second biodegradable polymer material comprising a diblock copolymer (PEG/PLGA) of polyethylene glycol (PEG) and poly(D,L-lactide-co-glycolide) (PLGA) applied to the primer layer.

### PRIORITY CLAIM

This patent application claims priority to German Patent Application No. 10 2008 006 455.6, filed Jan. 29, 2008, the disclosure of which is incorporated herein by reference in its entirety.

### FIELD

The present disclosure relates to an implant having a base body comprised entirely or in part of a biocorrodible metallic material wherein at least the parts of the base body made of the biocorrodible metallic material are covered with a corrosion-inhibiting coating. The coating comprises a primer layer of a first biodegradable polymer material and a protective layer of a second biodegradable polymer material applied to the primer layer.

### BACKGROUND

In modern medical technology, implants are used in a variety of ways. Implants are used for supporting blood vessels, hollow organs and duct systems (endovascular implants), for fastening and temporary fixation of tissue implants and tissue transplants, and also for orthopedic purposes, e.g., as nails, plates or screws.

Implantation of stents is one of the most effective therapeutic measures for treatment of vascular diseases. Stents provide a supporting function in a patient's hollow organs. Stents of a traditional design have a filigree supporting structure of metallic struts which are initially in a compressed form for introduction into the body and are dilated at the site of application. One of the main areas of application of such stents is for permanently or temporarily dilating vasoconstrictions, in particular, constrictions (stenoses) of the coronary vessels, and maintaining vascular patency. In addition, there are also known aneurysm stents that support damaged vascular walls.

The base body of each implant, in particular, a stent, consists of an implant material. An implant material is a nonviable material that is used for an application in medicine and interacts with biological systems. The main prerequisite for use of a material as an implant material that comes in contact with the biological environment when used as intended is its biological compatibility (referred to as biocompatibility). For purposes of the present disclosure, biocompatibility means the ability of a material to induce an appropriate tissue reaction in a specific application. This includes an adaptation of the chemical, physical, biological and morphological surface properties of an implant to the recipient tissue to achieve a clinically-desired interaction. The biocompatibility of the implant material depends largely on the chronological course of the reaction of the biosystem into which the implant is implanted. Irritation and inflammation may occur in the relatively short term and may lead to tissue changes. Biological systems may thus react in various ways, depending on the properties of the implant material. According to the reaction of the biosystem, the implant materials may be subdivided into bioactive, bioinert and degradable/absorbable materials. Only degradable/absorbable metallic implant materials, which are also referred to below as biocorrodible metallic materials, are of interest for the purposes of the present disclosure.

The use of biocorrodible metallic materials is recommended, in particular, because often the implant need only remain in the body temporarily to fulfill the medical purpose. Implants of permanent materials, i.e., materials that are not degraded in the body, may optionally be removed again because rejection reactions of the body may occur in the medium range and long range even when there is a high biocompatibility.

One approach for preventing another surgical procedure is to form the implant entirely or in part of a biocorrodible metallic material. For purposes of the present disclosure, biocorrosion refers to processes that are caused by the presence of biological media and lead to a gradual degradation of the structure made of the material. At a certain point in time, the implant or at least the parts of the implant made of the biocorrodible material will lose their mechanical integrity. The degradation products are largely absorbed by the body. In the best case, e.g., in the case of magnesium, the degradation products even have a positive therapeutic effect on the surrounding tissue. Small quantities of unabsorbable alloy constituents are nontoxic and are tolerable.

Known biocorrodible metallic materials comprise pure iron and biocorrodible alloys of the main elements magnesium, iron, zinc, molybdenum and tungsten. German Patent Application No. 197 31 021 proposes, among other things, production of medical implants from a metallic material whose main constituent is an element from the group consisting of alkali metals, alkaline earth metals, iron, zinc and aluminum. Alloys based on magnesium, iron and zinc are described as being especially suitable. Secondary constituents of the alloys may include manganese, cobalt, nickel, chromium, copper, cadmium, lead, tin, thorium, zirconium, silver, gold, palladium, platinum, silicon, calcium, lithium, aluminum, zinc and iron. In addition, German Patent Application No. 102 53 634 also describes the use of a biocorrodible magnesium alloy containing magnesium >90%, yttrium 3.7-5.5%, rare earth metals 1.5-4.4% and the remainder <1%. This alloy is suitable, in particular, for the production of an endoprosthesis, e.g., in the form of a stent. Regardless of the progress that has been achieved in the field of biocorrodible metal alloys, the alloys known so far have only limited applicability because of their corrosion properties. In particular, the relatively rapid biocorrosion of magnesium alloys limits their possible use.

Traditional technical applications of molded bodies made of metallic materials, in particular, magnesium alloys, outside of medical technology usually require extensive suppression of corrosion processes. Accordingly, the goal of most technical methods for improving the corrosion behavior is to completely inhibit corrosion processes. In the present disclosure, however, the goal of improving the corrosion behavior of the biocorrodible metallic materials is not to completely suppress the corrosion processes, but instead to inhibit the corrosion processes temporarily. For this reason, most known measures for improving corrosion protection are not suitable. Furthermore, for a medical technical use, toxicological aspects must also be taken into account. Furthermore, corrosion processes depend greatly on the medium in which the corrosion processes take place. Therefore, it is not usually possible to transfer findings about the properties of specific anticorrosion prevention coatings, said findings obtained in a technical field under traditional environmental conditions, to processes in a physiological environment.

One known method for improving the corrosion behavior (in the sense of increasing corrosion protection) is to produce a corrosion-preventing layer on the molded body made of the metallic material. Known methods for producing a corrosion-preventing layer have been developed and optimized from the standpoint of a technical use of a coated molded object, but not for medical technical use in biocorrodible implants in a physiological environment. These known methods include, for example, applying polymers or organic top coats, producing an enamel, chemical conversion of the surface, hot gas oxidation, anodizing, plasma sputtering, laser beam fusion, PVD methods, ion implantation or lacquering.

European Patent Application No. 1 389 471 describes a stent having a base body of a biocorrodible metallic material, in particular, a magnesium alloy. The implant surface has a polymer coating of a high-molecular poly(L-lactide). A primer layer may be provided between the implant surface and the polymer coating.

German Patent Application No. 198 43 254 describes implants having a coating of a polymer mixture containing cyanoacrylate or methylene malonic ester. The polymer mixture may contain poly(D,L-lactide-co-glycolide).

One aspect of the present disclosure provides an improved or at least an alternative coating for an implant of a biocorrodible metallic material which produces a temporary inhibition but not complete suppression of the corrosion of the material in a physiological environment.

### SUMMARY

The present disclosure describes several exemplary embodiments of the present invention.

One aspect of the present disclosure provides an implant, comprising a) a base body consisting at least partially of a biocorrodible metallic material, wherein at least the parts of the base body made of the biocorrodible metallic material are substantially covered with a corrosion-inhibiting coating, the coating comprising a primer layer of a first biodegradable polymer material and a protective layer applied to the primer layer and further comprising a second biodegradable polymer material, wherein (i) the first biodegradable polymer material of the primer layer comprises a poly(D,L-lactide) (PDLLA) with a degree of polymerization in the range of 5 to 20; and (ii) the second biodegradable polymer material of the protective layer comprises a diblock copolymer (PEG/PLGA) of polyethylene glycol (PEG) and poly(D,L-lactide-co-glycolide) (PLGA).

It has been found that applying a coating of the aforementioned composition leads to the development of a protective layer that permanently, completely or largely inhibits corrosion in a physiological environment. In other words, in a physiological environment, the implant still undergoes corrosion but at a greatly retarded rate. It is also advantageous, in particular, that only top coats having a very small layer thickness of the polymer are required. This has the advantage that adaptation of proven geometries of the implant base body is usually eliminated, e.g., in the case of stents, even struts having a small diameter can be coated. This is due to a reduction in the amount of polymer on the implant so that, for this reason alone, adverse tissue reactions are prevented in comparison with traditional coatings.

The biocorrodible metallic material is preferably a biocorrodible alloy selected from the group of elements consisting of magnesium, iron, zinc, molybdenum and tungsten. The material is, in particular, a biocorrodible magnesium alloy. For purposes of the present disclosure, the term “alloy” means a metallic structure in which the main components are magnesium, iron, zinc, molybdenum or tungsten. The main component is the alloy component whose amount by weight in the alloy is the greatest. The amount of the main component is preferably more than 50 wt %, and, in more preferably, more than 70 wt %.

The magnesium alloy with the following composition is especially preferred: rare earth metals 5.2-9.9 wt %, including yttrium 3.7-5.5 wt % and remainder <1 wt %, whereby magnesium accounts for the remaining amount of the alloy up to a total of 100 wt %. This magnesium alloy has already confirmed in clinical trials its special suitability, i.e., the magnesium alloy has manifested a high biocompatibility, favorable processing properties and good mechanical characteristics. Through in vivo studies, it has been shown that the magnesium alloy is degraded and/or replaced by endogenous components. For purposes of the present disclosure, the collective term “rare earth metals” means scandium (21), yttrium (39), lanthanum (57) and the 14 elements that follow lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71). In addition, magnesium alloys containing up to 6 wt % zinc are also preferred. Also, a magnesium alloy with the composition yttrium 0.5-10 wt %, zinc 0.6-6 wt %, calcium 0.05-1 wt %, manganese 0-0.5 wt %, silver 0-1 wt %, cerium 0-1 wt % and zirconium 0-1 wt % or silicon 0-0.4 wt % is especially preferred, whereby the amounts are based on percent by weight (wt %) of the alloy, and magnesium as well as manufacturing-related impurities account for the remaining amount of the alloy up to 100 wt %.

The alloys of the elements magnesium, iron, zinc, molybdenum or tungsten are to be selected in their composition so that the alloys are biocorrodible. For purposes of the present disclosure, biocorrodible means alloys in which a degradation/rearrangement takes place in a physiological environment so that the part of the implant comprising the material is entirely or at least predominantly no longer present. The test medium used for testing the corrosion performance of an alloy in question is artificial plasma such as that specified according to published standard EN ISO 10993-15:2000 for biocorrosion tests (composition NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L). A sample of the alloy to be tested is, therefore, stored in a defined amount of the test medium at 37° C. in a sealed sample container. At intervals of a few hours up to several months, depending on the anticipated corrosion behavior, the samples are removed and tested for traces of corrosion by known methods. The artificial plasma according to EN ISO 10993-15:2000 corresponds to a blood-like medium and thus simulates an appropriate physiological environment.

The corrosion process can be quantified by stating a corrosion rate. A prompt degradation is associated with a high corrosion rate and vice versa. Based on the degradation of the entire molded body, a surface that has been modified according to the present disclosure will lead to a reduction in corrosion rate. In the case of coronary stents, the mechanical integrity of the structure should preferably be maintained over a period of three months or more after implantation.

For purposes of the present disclosure, implants are devices introduced into the body by a surgical procedure or a minimally invasive procedure and include fastening elements for bones, e.g., screws, plates or nails, surgical suture materials, intestinal clamps, vascular clips, prostheses in the area of the heart and soft tissue, e.g., stents and anchoring elements for electrodes, in particular, pacemakers or defibrillators. The implant consists entirely or in part of the biocorrodible material. If only part of the implant is made of the biocorrodible material, then this part is to be coated accordingly.

The implant is preferably a stent. Stents of a traditional design have a filigree structure of metallic struts which are initially present in an unexpanded state for introduction into the body and are dilated into an expanded state at the site of application. In the case of stents, there are special requirements of the corrosion-inhibiting layer. The mechanical load on the material during expansion of the implant has an influence on the course of the corrosion process and it may be assumed that the stress corrosion cracking is intensified in the areas under stress. A corrosion-inhibiting layer should take this circumstance into account. In addition, a hard corrosion-inhibiting layer could flake off during expansion of the stent and cracking in the layer during expansion of the implant could be unavoidable. Finally, the dimensions of the filigree metallic structure must be taken into account and, if possible, only a thin but uniform corrosion-inhibiting layer should be produced. It has now been found that applying the coating of the present disclosure meets these requirements entirely or at least largely.

Poly(D,L-lactide) (PDLLA) has a terminal carboxylic acid function which is known to be capable of forming carboxylates on magnesium surfaces. Bonding via carboxylates ensures the required bonding force of the primer layer on the implant surface as is necessary for the intended purpose. The poly(D,L-lactide) (PDLLA) of the primer layer preferably has a degree of polymerization of 12.

In addition, it is preferable if the diblock copolymer (PEG/PLGA) contains polymer blocks of polyethylene glycol (PEG) with a molecular weight in the range of 2000 to 8000 g/mol.

The polymer blocks of polyethylene glycol (PEG) preferably contribute 1% to 20% to the total weight of the diblock copolymer (PEG/PLGA).

Furthermore, it is preferable if the diblock copolymer (PEG/PLGA) has an inherent viscosity in the range of 0.5 to 2 dL/g (0.1%, CHCl₃, 25° C.). The inherent viscosity is especially preferably 1 dL/g.

According to another exemplary embodiment, the polymer blocks of poly(D,L-lactide-co-glycolide) (PLGA) have monomer ratios between 2:1 and 1:2, in particular, a monomer ratio of 1:1.

Finally, according to another exemplary embodiment, the implant is a stent and the amount of diblock copolymer (PEG/PLGA) applied is 15 to 20 μg per mm of stent length. For layer weights below the stated lower limit, homogeneous coverage of the areas of the base body to be coated is no longer ensured so that it is difficult to reproducibly establish the desired corrosion behavior. Above the aforementioned limit for the layer weight, inherent stresses may occur within the layer, leading to inhomogeneities which, in turn, may make it difficult to achieve a reproducible setting of the desired corrosion behavior. It is self-evident that the corrosion-inhibiting effect of the coating increases with an increase in the layer weight. To achieve a predefined corrosion behavior, those skilled in the art may proceed as described in one exemplary embodiment hereinbelow.

Sample bodies of the biocorrodible metallic material are produced and covered with a primer layer and then with a protective layer of a predefinable layer weight. In this way five test bodies, for example, can be produced with different layer weights, their corrosion behavior subsequently being quantified (e.g., by determination of the corrosion rate) and allowing a qualitative prediction of the relationship between layer weight and corrosion behavior. The resulting data for the corrosion behavior are compared with the desired corrosion behavior. If this comparison still shows significant deviations from each of the values obtained from the test samples, then starting from the most proximate value, the layer weight is varied in other test bodies. Ultimately those skilled in the art can determine a layer weight for the desired corrosion behavior by routinely working through this optimization procedure.

### BRIEF DESCRIPTION OF THE DRAWINGS

Various aspects of the present disclosure are described hereinbelow with reference to the accompanying figures.

FIG. 1 shows the degradation behavior of a stent modified according to one exemplary embodiment of the present disclosure in comparison with an uncoated stent, a stent covered only with a primer layer and a stent covered with a primer layer and a protective layer; and

FIG. 2 shows the degradation behavior of a stent modified according to the present disclosure as a function of the layer weight of the protective layer applied.

### DETAILED DESCRIPTION

Various nonlimiting exemplary embodiments of the present invention are disclosed in the following examples.

### EXAMPLES

### General Procedures

In the following exemplary embodiments, the coating is described of stents made of the commercially available magnesium alloy WE43 (according to ASTM) with a rare earth metal content of approximately 3 wt %, not including yttrium, and an yttrium content of approximately 4 wt % as well as a stent length of 10 mm.

### Producing the Primer Layer

According to a first exemplary embodiment, a suspension was prepared of 200 mg poly(D,L-lactide) with a degree of polymerization of 12 (available under the brand name L 104™ from the company Boehringer Ingelheim) in 10 mL absolute ethanol. The stent was incubated in this suspension for 60 hours at room temperature, then removed, rinsed with absolute ethanol and dried in air.

According to a second exemplary embodiment, a solution was prepared of 200 mg poly(D,L-lactide) with a degree of polymerization of 12 (available under the brand name Resomer L 104™ from the company Boehringer Ingelheim) in 10 mL absolute chloroform. The stent was incubated in this suspension for 60 hours at room temperature, then removed and dried in air.

### Producing the Protective Layer

A 0.1 wt % solution was prepared of diblock copolymer (PEG/PLGA) of polyethylene glycol (PEG) and poly(D,L-lactide-co-glycolide) (PLGA) in absolute acetone, in which the diblock copolymer (PEG/PLGA) contained polymer blocks of polyethylene glycol (PEG) with a molecular weight in the range of 5000 g/mol, the polymer blocks of polyethylene glycol (PEG) contributed 5% to the total weight of the diblock copolymer (PEG/PLGA) and the polymer blocks of poly(D,L-lactide-co-glycolide) (PLGA) had a monomer ratio of 1:1 (obtainable under the brand name Resomer RGP d 5055™ from the company Boehringer Ingelheim). The solution was sprayed onto one of the stents to be coated and then was dried in air.

### Example 1

### Stent With a Primer Layer and a Protective Layer

A stent produced by the method described hereinabove with a primer layer was then coated with a protective layer according to the procedure described hereinabove. The spraying operation was repeated until the weight of the applied protective layer was 75 μg.

### Example 2

A stent with a primer layer and a protective layer was produced in the same way as described in Example 1. However, the spraying operation was repeated until the weight of the applied protective layer was 200 μg.

### Example 3

A stent with a primer layer and a protective layer was produced in the same way as described in Example 1. However, the spraying operation was repeated until the weight of the applied protective layer was 350 μg.

### Comparative Example 1

### Stent Without a Coating

A stent without a coating was used for comparison purposes.

### Comparative Example 2

### Stent With a Primer Layer

A stent with a primer layer created according to Example 2 described hereinabove was produced for comparison purposes.

### Comparative Example 3

### Stent With a Primer Layer and a Noninventive Biocorrodible Protective Layer

For comparison purposes, a stent was produced with a primer layer produced by the method described hereinabove and an alternative protective layer in comparison with the layer of the present disclosure. This protective layer consists of a low-molecular poly(D,L-lactide-co-glycolide) PLGA 85:15, with an inherent viscosity of 0.63 dL/g and was applied in the same way as in production of the protective layer of the present disclosure.

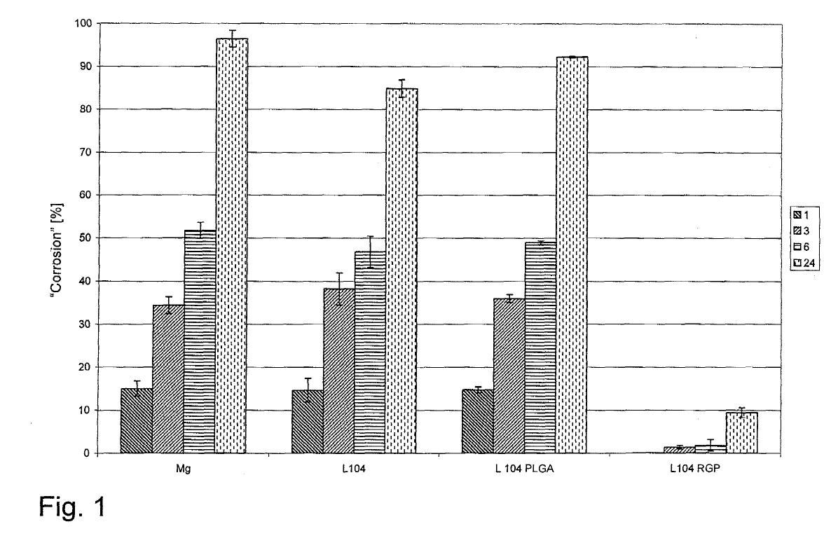

FIG. 1 illustrates the extent of the corrosion of the stent according to Example 1 (designated as L104 RGP in the figure) and Comparative Examples 1 to 3 (designated as Mg, L104, L104 PLGA in the figure). To do so, the stents were incubated for 1, 3, 6 and 24 hours in a PBS solution (phosphate-buffered saline solution with the composition: 8 g NaCl, 0.2 g KCl, 1.44 g Na₂HPO₄, 0.24 g KH₂PO₄ in 1 L H₂O, adjusted to pH 7.4). As can be seen, a combination of a primer layer and the protective layer of the present disclosure leads to a substantial inhibition of the corrosion of the stent but does not suppress the process completely. The extent of the corrosion is such that the supporting force of the stent is still largely preserved even after 24 hours.

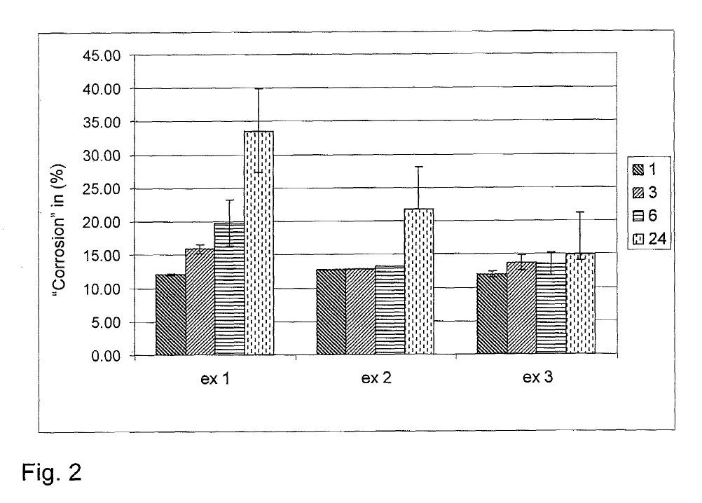

FIG. 2 shows the influence of the layer weight on the extent of the corrosion in a PBS solution after 1, 3, 6 and 24 hours. The stents from Examples 1 to 3 are compared (designated as ex1, ex2, ex3 in the figure). As this shows, the corrosion behavior can be influenced by varying the weight of material applied for the protective layer. The greater the weight applied, the longer the delay in corrosion.

All patents, patent applications and publications referred to herein are incorporated by reference in their entirety.

What is claimed is:

1. An implant, comprising:
a) a base body consisting at least partially of a biocorrodible metallic material, wherein at least the parts of the base body made of the biocorrodible metallic material are substantially covered with a corrosion-inhibiting coating, said coating comprising a primer layer of a first biodegradable polymer material and a protective layer applied to the primer layer and further comprising a second biodegradable polymer material, wherein
(i) the first biodegradable polymer material of the primer layer comprises a poly(D,L-lactide) (PDLLA) with a degree of polymerization in the range of 5 to 20; and
(ii) the second biodegradable polymer material of the protective layer comprises a diblock copolymer (PEG/PLGA) of polyethylene glycol (PEG) and poly(D,L-lactide-co-glycolide) (PLGA).

2. The implant of claim 1, wherein the biocorrodible metallic material is a biocorrodible alloy selected from the group consisting of magnesium, iron, zinc, molybdenum and tungsten.

3. The implant of claim 2, wherein the biocorrodible metallic material is a magnesium alloy.

4. The implant of claim 1, wherein the implant is a stent.

5. The implant of claim 1, wherein the poly(D,L-lactide) (PDLLA) of the primer layer has a degree of polymerization of 12.

6. The implant of claim 1, wherein the diblock copolymer (PEG/PLGA) contains polymer blocks of polyethylene glycol (PEG) with a molecular weight in the range of 2000 to 8000 g/mol.

7. The implant of claim 1, wherein the polymer blocks of polyethylene glycol (PEG) comprise 1% to 20% to the total weight of the diblock copolymer (PEG/PLGA).

8. The implant of claim 1, wherein the diblock copolymer (PEG/PLGA) has an inherent viscosity in the range of 0.5 to 2 dl/g (0.1% in chloroform at 25° C.).

9. The implant of claim 1, wherein the polymer blocks of poly(D,L-lactide-co-glycolide) (PLGA) have monomer ratios between 2:1 and 1:2.

10. The implant of claim 9, wherein the polymer blocks of poly(D,L-lactide-co-glycolide) (PLGA) have a monomer ratio of 1:1.

11. The implant of claim 4, wherein the implant is a stent and the applied amount of the diblock copolymer (PEG/PLGA) is 15 to 20 μg per mm of stent length.
